(19) United States
(12) Patent Application Publication
(10) Pub. No.: US 2007/0277834 A1
(43) Pub. Date: Dec. 6, 2007

(54) ABDOMEN COVER

(76) Inventor: K-Wan PARK, Seoul (KR)

Correspondence Address: Joseph Hyosuk Kim, Ph.D.;JHK Law, P.O. Box 1078, La Canada, CA, 91012-1078, US

(21) Appl. No.: 11/838,841

(22) Filed: Aug. 14, 2007

Related U.S. Application Data
(62) Division of application No. 10/888,142, filed on Jul. 8, 2004.

(30) Foreign Application Priority Data
Dec. 18, 2003 (KR) 2003-39482
Feb. 11, 2004 (KR) 2004-3417

(51) Int. Cl. A61F 13/14

(52) U.S. Cl. 128/846

(57) ABSTRACT

The present invention discloses an abdomen cover that is covered on a wearer's abdominal region to protect it while he or she is asleep especially in summer, thereby preventing having the trouble in his abdomen, that is also used during the day or during working outside for long hours, thereby keeping his abdominal region in a warm state, that is designed to conform to a wearer's body shape, thereby maintaining the fixed position thereof on his abdominal region without any displacement, and that is formed integrally with all kinds of clothes or detachably coupled to them in an easy manner. The abdomen cover includes a cover body having an inclined portion slantingly formed at both sides thereof to be conform to a wearer's body shape; and a fastening band formed at the end portion of one side or each of both sides of the cover body, for tying the cover body around the wearer's abdominal region.

### RELATED APPLICATIONS

This application is a divisional of U.S. patent application Ser. No. 10/888,142, filed Jul. 8, 2004, which claims benefit of priority from Korean Patent Applications 2004-3417, filed Feb. 11, 2004, and 2003-39482, filed Dec. 18, 2003. All three applications are hereby incorporated by reference as if fully set forth.

### FIELD OF THE INVENTION

The present invention relates to an abdomen cover, and more particularly, to an abdomen cover that is covered on a wearer's abdominal region to protect it while he or she is asleep especially in summer, thereby preventing having the trouble in his abdomen, that is also used during the day or during working outside for long hours, thereby keeping his abdominal region in a warm state, that is designed to conform to a wearer's body shape, thereby improving a feeling of wearing and maintaining the fixed position thereof on his abdominal region without any displacement, and that is formed integrally with all kinds of clothes or detachably coupled to them in an easy manner.

### BACKGROUND OF THE RELATED ART

In recent days, it is in hot summer common that a temperature does not fall at even night, stopping at a predetermined level, which feels like that people still live in a tropical climate. Especially, children or old and weak people do not sleep well in such the climate. As a result, they do not sleep under a coverlet, and they also sleep in a state of being undressed, while exposing their abdominal region to the outside, which may cause the generation of trouble in their abdomen.

In hot summer, even though they sleep under the coverlet, they generally push it under their foot, or even though they sleep while dressing their underwear, they take off it during sleep. Unfortunately, there has been proposed no abdomen cover for covering and protecting a wearer's abdominal region while he or she is asleep.

To overcome the above-mentioned problems, the present inventor has proposes an abdomen cover (which is disclosed in Korean Utility Model Registration No. 296817 (registered on Nov. 21, 2002). As shown in FIG. 1, the conventional abdomen cover includes a cover body 10 adapted to cover a wearer's abdominal region and a pair of upper and lower fastening bands 20 and 30 that are provided at both sides of the cover body 10, respectively. In this case, the size of the cover body 10 can be adjusted to conform to the wearer's body shape.

The upper and lower fastening bands 20 and 30 are formed in such a manner that one side bands 21 and 31 are relatively longer than the other side bands 22 and 32.

FIG. 2 is a view showing the way of using the conventional abdomen cover, wherein the upper and lower fastening bands 20 and 30 are tied around the wearer's abdominal region. Since one side bands and the other side bands have different lengths, they are placed at the side regions, but not at the wearer's back region, which prevents the wearer's back during sleep from hurting by the ties.

Also, the upper and lower fastening bands 20 and 30 are positioned at the places separated by a predetermined distance from the corners of the cover body 10. This prevents the cover body 10 from being separated from the wearer's abdominal region.

It is however found that the abdomen cover itself is moved upwardly during the wearer's sleep. The reason for this is because the upper and lower fastening bands 20 and 30 are spaced apart from each other by a predetermined interval. Thus, the abdomen cover is not positioned at the wearer's abdominal region and is moved with a result of abutting against the floor of bed. Thus, the conventional abdomen cover is not fixedly placed on the wearer's abdominal region.

Moreover, due to the separation of the upper fastening band 20 and the lower fastening band 30 there is a gap between the conventional abdomen cover and the wearer's abdominal region, and since the cover body 10 takes a generally rectangular shape, the conventional abdomen cover does not conform to the wearer's body shape, which makes the wearer feel uncomfortable when he or she wears it.

### SUMMARY OF THE INVENTION

Accordingly, the present invention has been made in view of the above-mentioned problems occurring in the prior art.

An object of the present invention is to provide an abdomen cover that is designed to conform to a wearer's body shape, thereby making the wearer feel comfortable when he or she wears it, that is fixedly placed on a wearer's abdominal region, without any displacement, and that is formed integrally with all kinds of clothes or detachably coupled to them in an easy manner.

To accomplish the above object, according to a first embodiment of the present invention, there is provided an abdomen cover covered on a wearer's abdominal region to protect the wearer's abdominal region, including: a cover body having an inclined portion slantingly formed at both sides thereof to be conform to a wearer's body shape; and a fastening band formed at the end portion of one side or each of both sides of the cover body, for tying the cover body around the wearer's abdominal region.

The cover body further includes an extended portion formed at the upper or lower portion thereof in such a manner as to be protruded upwardly or downwardly from the upper or lower portion thereof.

The extended portion has a gently curved shape, a heart-like shape, an angled shape, or a shape of “ [symbol] ” or “ [symbol] ”.

The extended portion formed at the lower portion of the cover body is extended downwardly to cover the groin and private region of the wearer.

The fastening band includes a single band integrally coupled to the cover body.

The fastening band or the cover body has any one of a detachable Velcro tape, a snap, a hook and a button and buttonhole which are formed at the end portion thereof in such a manner as to have a male and female pair.

The button is a toggle button, and the fastening band or the cover body is provided with a coupling loop into which the toggle button is fastened, at the end portion thereof.

The fastening band is formed of any one of fabric, a rubber material, and a mixture of the fabric with the rubber material.

The fastening band is covered with the fabric around the outer surface thereof and is embedded with the rubber material in the inside thereof.

The fastening band has a folded portion provided on the outer surface thereof, for applying an elastic force thereto.

The cover body is provided with a pair of auxiliary support bands at one side or each of the both end portions thereof in such a manner as to be met and coupled to the fastening band.

The auxiliary support band is coupled to the edge portion of the cover body at one end thereof in such a manner as to be coupled to the fastening band.

The cover body is provided with a reinforced member along a circumferential direction thereof.

The cover body is provided with a heating member that includes any one of cotton, fabric, and a plant on the inside thereof.

To accomplish the above object, according to a second embodiment of the present invention, there is also provided an abdomen cover covered on a wearer's abdominal region to protect the wearer's abdominal region, including: a cover body having detachable means formed at one side thereof, to be detachably coupled to wearer's clothes; and a fastening band formed at the end portion of one side or each of both sides of the cover body.

The detachable means has any one of a detachable Velcro tape, a snap, a hook and a button which are formed in such a manner as to have a male and female pair.

To accomplish the above object, according to a third embodiment of the present invention, there is provided an abdomen cover covered on a wearer's abdominal region to protect the wearer's abdominal region, including: a cover body coupled integrally to clothes at one side thereof; and a fastening band formed at the end portion of one side or each of both sides of the cover body.

The clothes include any one of pants, skirts, shirts, pajamas, panties, brassieres and vests.

The cover body is cut along a longitudinal direction at a central portion thereof such that it is openable to the left and right sides, and the cover body is provided with locking means at the central portion thereof.

The cover body is provided with a pocket at one face thereof, and the pocket has locking means formed at the inside of the top portion thereof.

Each of the locking means at the central portion of the cover body and the locking means at the inside of the top portion of the pocket of the cover body has any one of a detachable Velcro tape, a snap, a button and a zipper which are formed in such a manner as to have a male and female pair.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a perspective view of a conventional abdomen cover;

FIG. 2 is a view showing the way of using the conventional abdomen cover;

FIG. 3 is a perspective view of an abdomen cover according to an embodiment of the present invention;

FIGS. 4 to 10 are perspective views of abdomen covers according to another embodiments of the present invention;

FIG. 11 is a view showing the edge portion member provided along the edge portion of a cover body of the abdomen cover of the present invention;

FIG. 12 is an enlarged sectional view showing another cover body of the abdomen cover of the present invention, wherein cotton or fabric pad is provided on the inside of the cover body;

FIG. 13 is a view showing the way of using the abdomen cover of the present invention; and

FIGS. 14 to 20 are front views showing abdomen covers according to still another embodiments of the present invention.

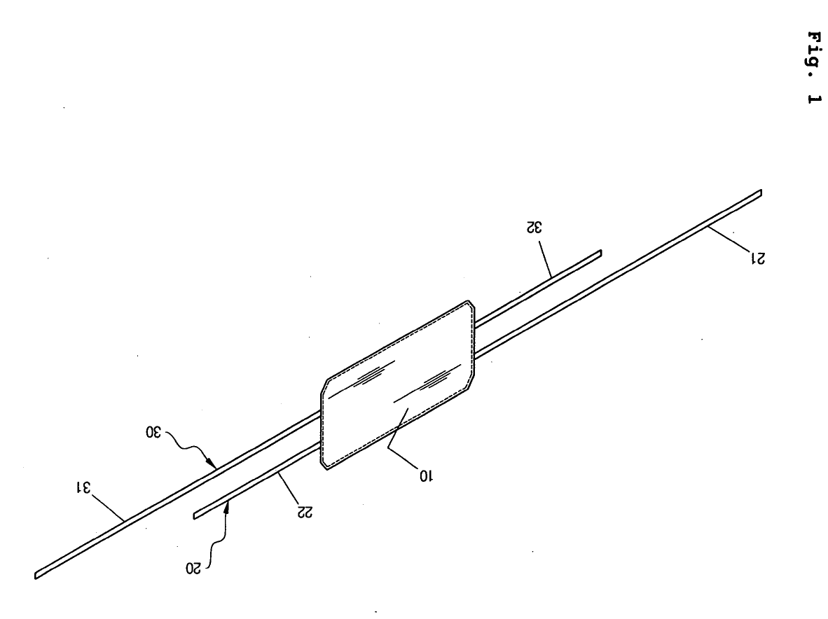

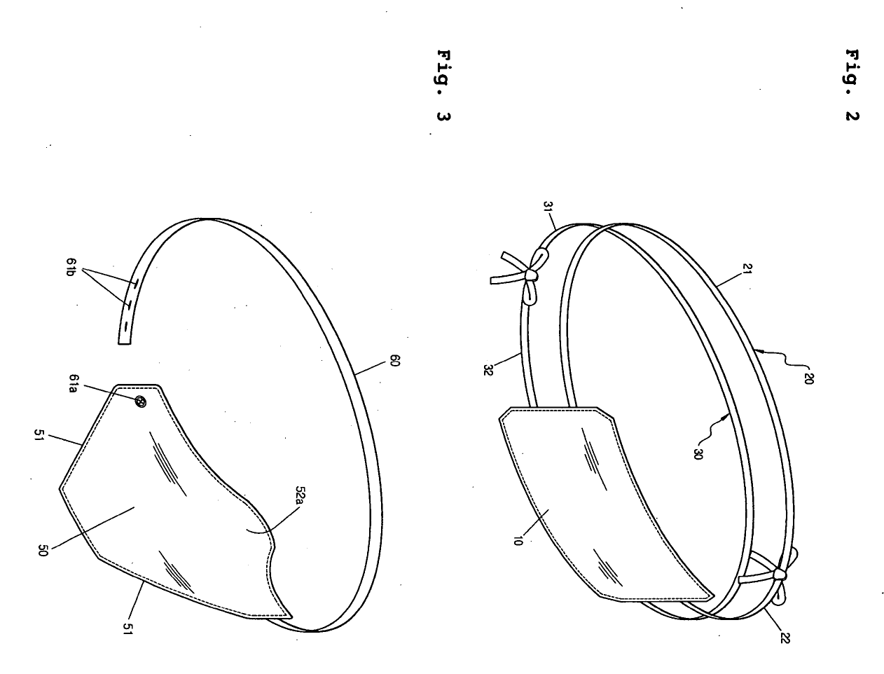

### DETAILED DESCRIPTION OF THE PREFERRED EMBODIMENT

Reference will now be made in detail to the preferred embodiments of the present invention, examples of which are illustrated in the accompanying drawings.

As shown in FIG. 3, an abdomen cover according to the present invention includes a cover body 50 that has a predetermined dimension, for covering a wearer's abdominal region, and a fastening band 60 that is provided at one side of the cover body 50 in such a manner as to be coupled to the cover body 50 around a wearer's waist.

The cover body 50 has an inclined portion 51 that is somewhat inclined toward a central portion thereof, at both sides thereof, respectively, and when the wearer puts on the abdomen cover of the present invention, the inclined portions 51 are placed contacted with a wearer's groin between the two legs, which makes him feel comfortable while he is wearing the abdomen cover.

The cover body 50 further includes an extended portion 52a, 52b, 52c, 52d, 52e and/or 52f that are protrudedly extended from the upper and/or lower portions thereof. The extended portions 52a, 52b, 52c and 52d that are extended from the upper portion of the cover body 50 serve to cover the stomach region of the wearer.

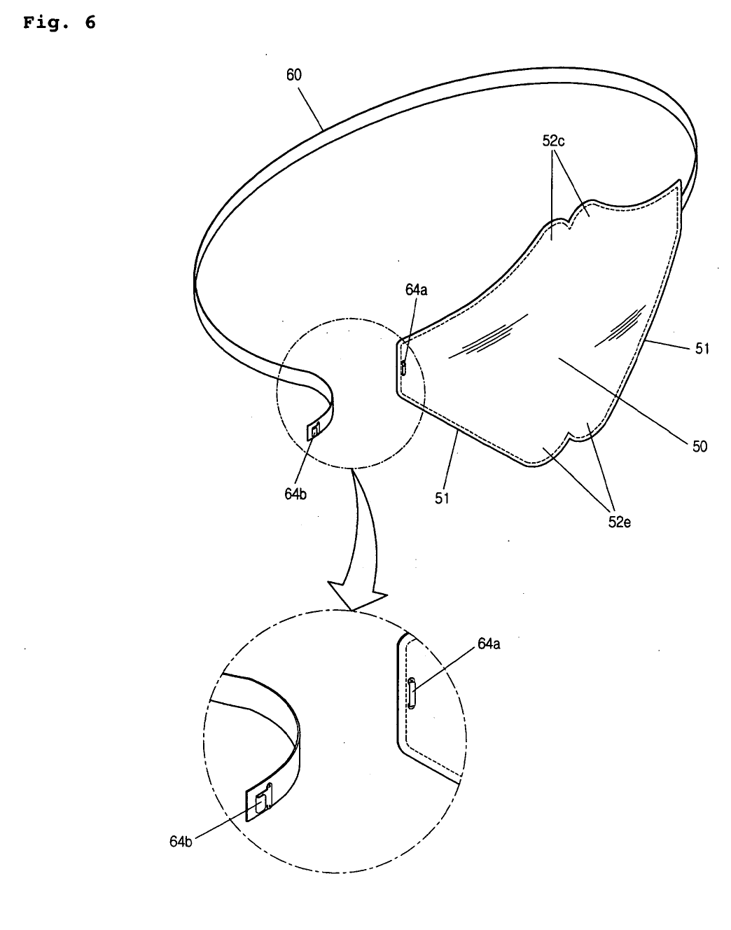

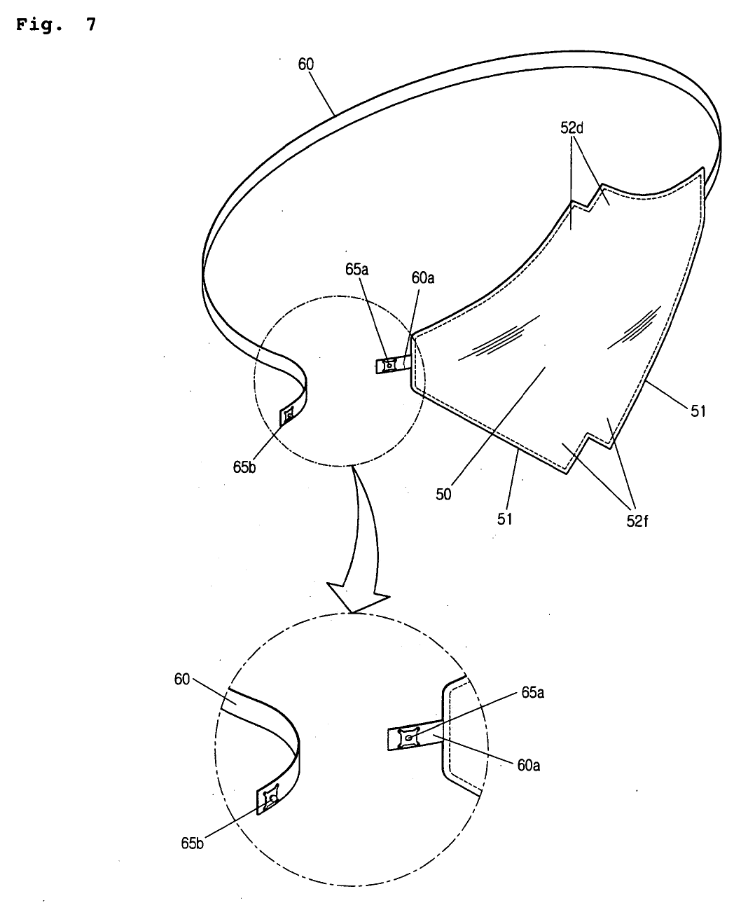

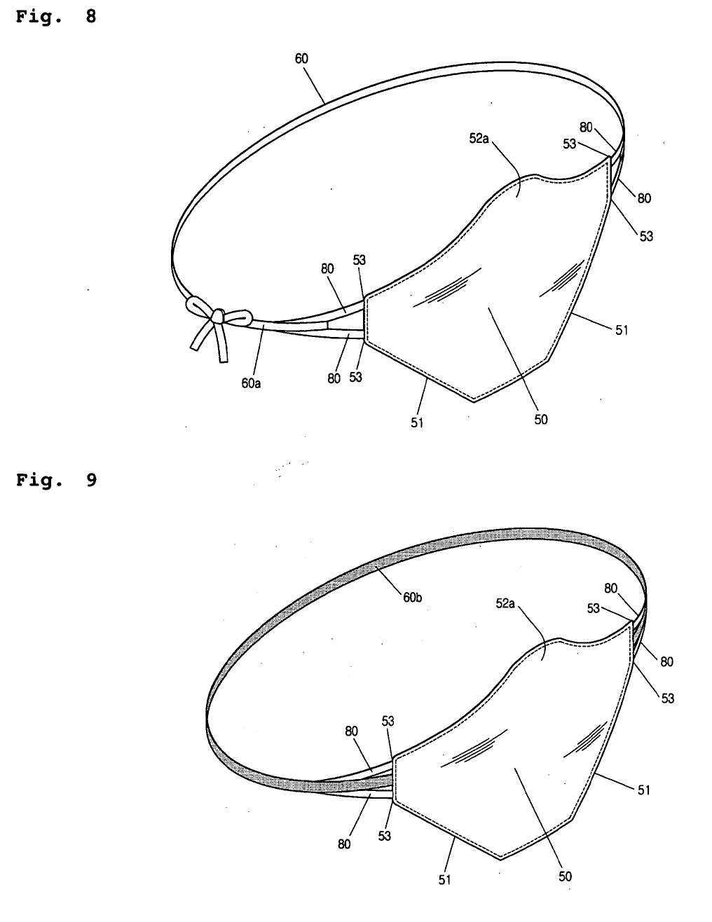

The extended portions 52a, 52b, 52c and 52d are designed to have a variety of shapes in accordance with wearer's preferences and outer appearances, and FIG. 3 shows the extended portion 52a that has a gently curved shape, and FIG. 5 shows the extended portion 52b that has an angled shape. FIG. 6 shows the heart-like extended portion 52c, and FIG. 7 shows the “ [symbol] ” shaped extended portion 52d.

FIGS. 6 and 7 show the heart-like extended portion 52e and the “ [symbol] ” shaped extended portion 52f that are extended from the lower portion of the cover body 50, which correspond to the upper extended portions 52c and 52d thereof. Otherwise, the extended portions may be designed to have another variety of shapes like stars, flowers, diamonds, and the like, which makes children and women develop the closeness toward the abdomen cover of this invention and also makes the outer appearance look more beautiful. When the wearer puts on the abdomen cover, thus, he does not feel a sense of rejection and he is not reluctant to prepare the abdomen cover as a present.

On the other hand, as shown in FIGS. 3 to 7, the fastening band 60 is formed at the end portion of one side of the cover body 50 in such a manner as to be detachably coupled at the end portion of the other side of the cover body 50. As shown in FIG. 8, the fastening band 60 is provided at the end portions of the both sides of the cover body 50, respectively, wherein one side fastening band is generally longer than the other side fastening band 60a, with a result that the tie is placed at the side of a wearer's waist.

In more detail, as shown in FIG. 3, the cover body 50 and the fastening band 60 are provided with a button 61a and one or more buttonholes 61b that are fastened to each other, at the end portions thereof. In order to conform to a wearer's body shape, therefore, the button 60a and one of the buttonholes 61b are fastened and fixedly fit around the wearer's waist.

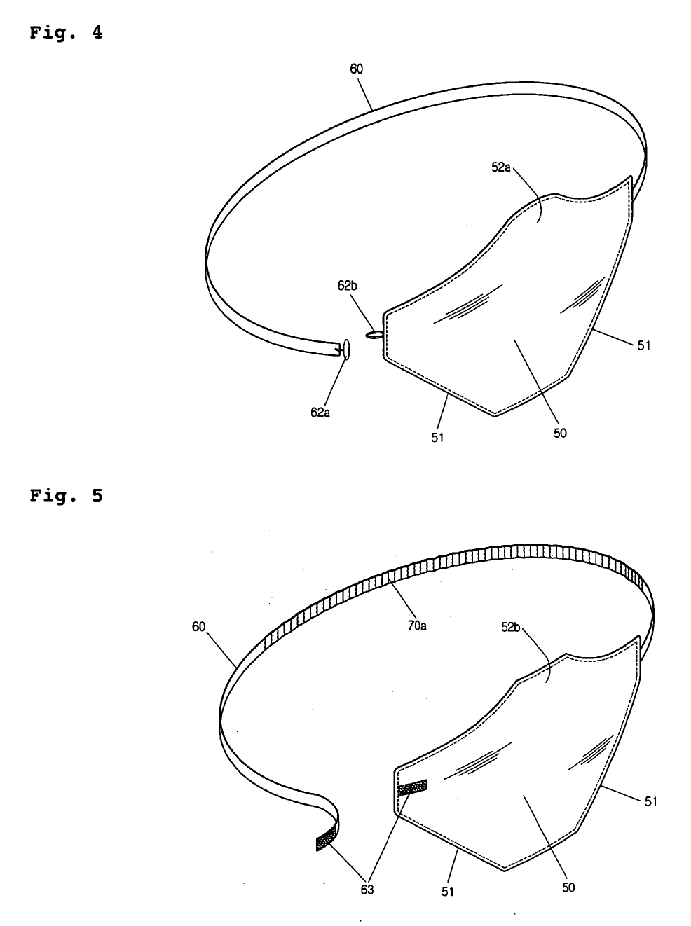

As shown in FIG. 4, the button 60a is formed of a toggle button 62a, and there is a coupling loop 62b that is fastened to the toggle button 62a at the end portion of the cover body 60 corresponding to the toggle button 62a. In this way, the button 62a can be easily fastened to the coupling loop 62b and unfastened therefrom, and therefore, this coupling way is adequate to old people and children that have a poor hand at handling the button.

According to another embodiments of the present invention, as shown in FIGS. 5 to 7, the fastening band 60 and the cover body 50 include a detachable Velcro tape 63, hooks 64a and 64b, and snaps 65a and 65b that are coupled to each other at the end portions thereof in such a manner as to have a male and female pair.

In case of the hooks 64a and 64b, as shown in FIG. 6, the hook 64a that is formed at one end portion of the fastening band 60 is a loop, and the hook 64b that is formed at one side of the cover body 50 is a locker that is detachably coupled to the loop 64a.

In case of the snaps 65a and 65b, as shown in FIG. 7, the snap 65a that is formed at one side fastening band 60a extended from the one side of the cover body 50 is a concave portion, and the snap 65b that is formed at the one end portion of the fastening band 60 is a protruded portion that is detachably coupled to the concave portion 65a.

At this time, the loop 64b and the locker 64a of the hook and the concave portion 65a and the protruded portion 65b of the snap are placed at the end portions of the fastening bands 60 and 60a and the one side of the cover body 50, without any limitation of the positions only if they correspond to each other.

As shown in FIGS. 8 to 10, the fastening band 60 may be formed at the both end portions of the cover body 50, respectively, and in this case, one side fastening band is relatively longer than the other side fastening band or has the same length as that.

In more detail, in FIG. 8 the fastening bands 60 and 60a that have different lengths from each other are tied to each other, and as shown, the tie is placed at the side of the wearer's waist, such that while he is asleep, he does not feel any hurt on his back.

On the other hand, as shown in FIG. 10, fastening bands 60c and 60d are extended to such a length that they are tied to each other at the front of the wearer. However, the invention is not limited to the positions of the tie as mentioned above, which may be varied freely in accordance with a wearer's convenience.

As shown in FIG. 8, there are auxiliary support bands 80 that are formed at both end portions of the cover body 50, wherein they are fixed on the cover body 50 at one ends thereof and coupled on the fastening bands 60 and 60a. In this case, only the auxiliary support bands 80 are coupled to the cover body 50, and the ends of the auxiliary support bands 80 are met and extended thus to form the fastening belts 60 and 60a.

As shown in FIG. 9, a fastening band 60b itself is coupled to the cover body 50. In this case, the auxiliary support bands 80 are coupled to the cover body 50 at one ends thereof and coupled to the fastening band 60b at the other ends thereof.

In this case, the auxiliary support bands 80 are preferably fixedly coupled at the both side corner portions of the cover body 50 in such a manner as to be coupled to the fastening bands 60, 60a and 60b. This prevents the cover body 50 from being separated from the wearer's abdominal region while he is wearing it.

The fastening band 60b according to the present invention as shown in FIG. 9 is formed integrally with the cover body 50. At that time, the auxiliary support bands 80 are coupled to the cover body 50, together with the fastening band 60b and the cover body 50, or it may be not provided thereto. The fastening band 60b is formed of a rubber band or a spandex material that exhibits good expanding and elastic features, which is fit around the wearer's waist in a very easy manner.

According to the abdomen cover of this invention, moreover, the fastening bands 60, 60a, 60b, 60c and 60d are fit around the wearer's waist. If he lies on his bed to sleep, generally, his back region is somewhat separated from the floor of the bed. Thus, since the fastening band is placed at the space that is formed between his back and the floor of the bed, the contact area between his back and the floor of the bed becomes small, and even though he changes his position on his bed during sleep, the cover body itself is not moved.

Additionally, the auxiliary support bands 80 are provided at the upper and lower portions of the fastening bands 60, 60a, 60b, 60c and 60d, being separated at a predetermined interval from each other, such that if the fastening bands 60, 60a, 60b, 60c and 60d are pulled and tied to each other, the auxiliary support bands 80 are also pulled and maintained at a tense state. Therefore, the cover body 50 is pulled from one to three positions at each of the both sides thereof, such that it cannot be loose at the both sides thereof, which makes the wearer feel comfortable while he is wearing the abdomen cover of this invention.

The fastening bands 60, 60a, 60b, 60c and 60d and the auxiliary support bands 80 are made of an elastic rubber band, general fabric, or the mixture of both of them. In other words, they may be formed of the rubber band itself, or is covered with fabric on the outer surface thereof and is embedded with the rubber band in the inside thereof.

The cover body 50 is made of an expandable spandex material, or may be formed of fabric. In addition, it is made of all kinds of functional or natural fabrics.

The cover body 50 has a variety of sizes to be adaptable to a wearer's body shape. In other words, it is divided into ones for adults or children. For the use of children or women, especially, it is wholly rounded, without any angled portion.

The cover body 50 is provided with a variety of colors and patterns on the surface thereof. Also, the cover body 50 may have all kinds of materials, colors, patterns, and shapes in accordance with the objects of using the abdomen cover like adults, children, women and men and the purposes of using it.

As shown in FIG. 5, the fastening band 60 is provided with a predetermined folded portion 70a that is adapted to give elasticity to the outer surface thereof, thereby improving the closeness to the wearer's waist. In this case, the folded portion 70a is formed over the fastening band 60 or on a part of the fastening band 60, and even though not shown in the drawing, it may be formed on the auxiliary support band 80.

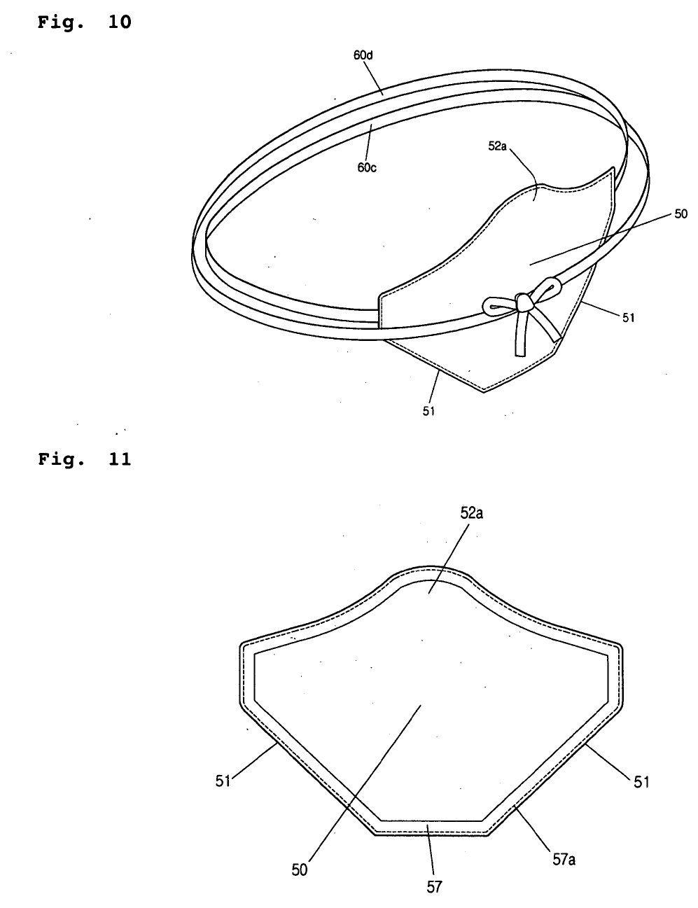

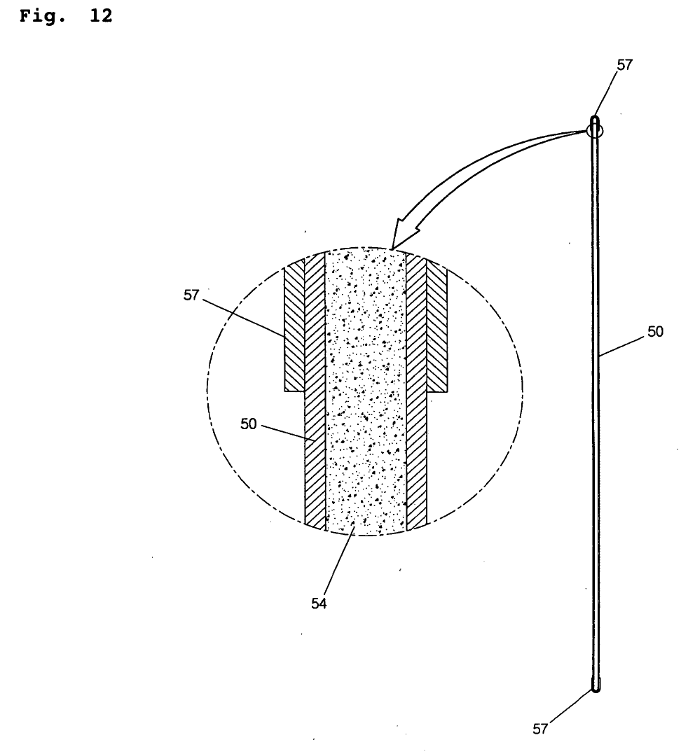

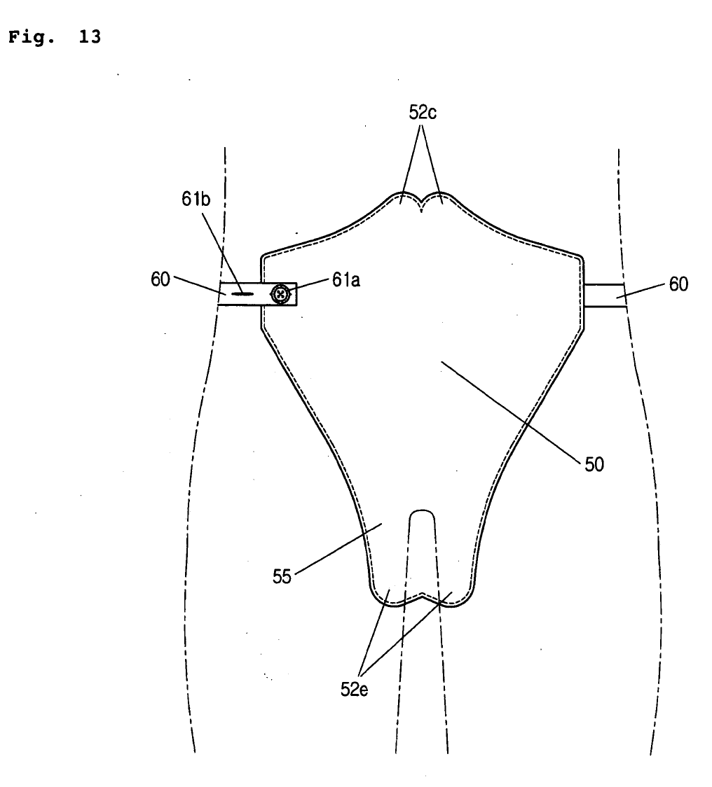

According to the present invention, as shown in FIG. 11, the cover body 50 has a reinforced member 57 that is formed of fabric sewn along a circumferential direction of the cover body 50, which makes the outer appearance look beautiful and also makes the abdomen cover finished rigidly. The reinforced member 57 is provided with a variety of embroidery designs, prints, letters, and so on.

A sewn line 57a is formed by stitching the inside of the edge portion of the cover body 50, such that the sewn line 57a is not exposed to the outside.

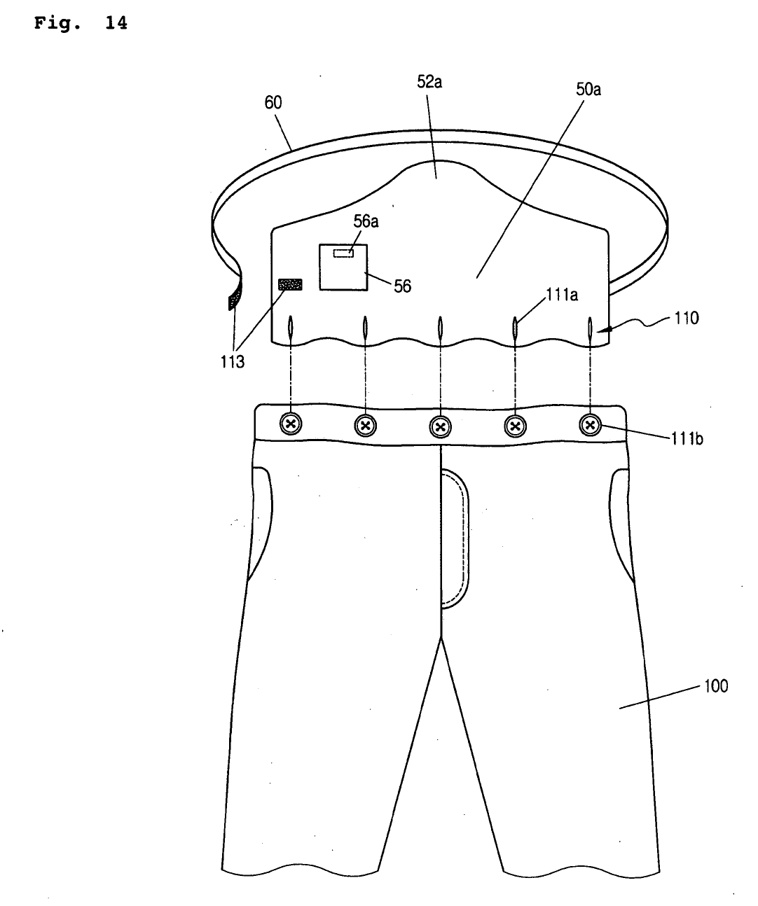

According to the present invention, as shown in FIG. 12, the cover body 50 is provided with a heating member 54 that is formed at the inside thereof. The heating member 54 is formed by selecting any one of cotton, fabric (inclusive of functional fabric), and a plant (e.g., leaf, hub, and the like). In this case, it is very helpful to people who work outside for many hours or it keeps the wearer's abdominal region at a warm state.

FIG. 12 shows the structure of the cover body 50 in which the edge portion member 57 is formed along the edge portion of the cover body 50, as shown in FIG. 10. Even in case of no edge portion member 57, of course, the structure of the cover body 50 as shown in FIG. 12 can be formed.

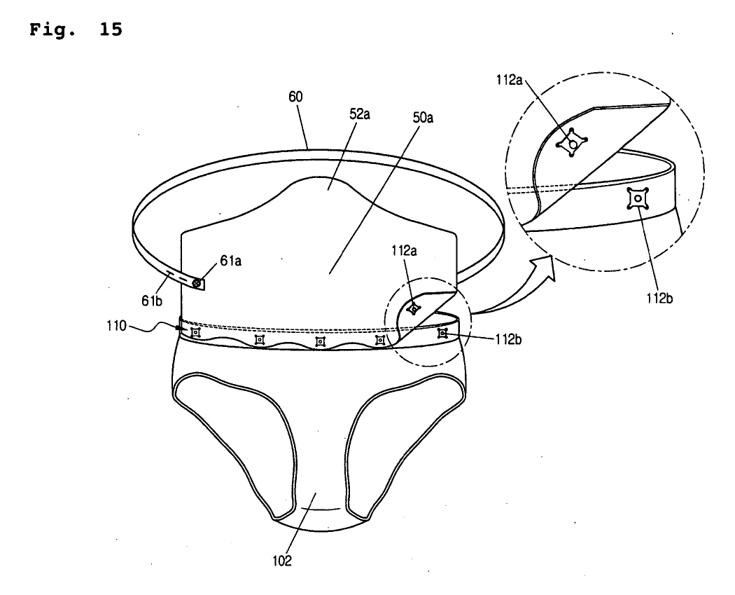

According to another embodiment of the present invention, as shown in FIG. 13, the cover body 50 is provided with an extended portion 55 that is extended downwardly from the lower portion thereof, for covering the groin between the wearer's two legs. As a result, the abdomen cover of this invention can cover the important regions of the human body like the groin and private region as well as the abdominal region especially in summer.

In this case, the downward extended portion 55 that is formed on the cover body 50 has a heart-like extended portion 53a extended from the bottom end thereof to the both sides thereof, respectively, and the cover body 50 also has another heart-like extended portion 52c extended from the top end thereof to the both sides thereof, respectively, corresponding to the extended portion 53a.

In a case where the abdomen cover of this invention is worn outside wearer's clothes, it functions as a simple blanket for drivers, workers who work outside for many hours, guards, passengers, workers who work at night, and so on. In this case, the cover body 50 has relatively large dimension and thickness, which of course gives a high heating efficiency to the wearer.

According to the present invention, as shown in FIG. 14, there is provided an abdomen cover including: a cover body 50a having detachable means 110 formed at one side thereof, the detachable means 110 being detachably coupled to wearer's clothes; and a fastening band 60 formed at both sides of the cover body 50, respectively, for tying the cover body 50a around a wearer's abdominal region.

In other words, the abdomen cover of this invention is worn by itself or outside wearer's clothes by means of the detachable means, and as shown in FIG. 14, the cover body 50a is detachably coupled to the wearer's clothes by the use of the detachable means 110.

A pair of pants 100 to which the cover body 50a is detachably attached are provided with one or more buttons 111b at the top end thereof, and to correspond with the buttons 111b, the cover body 50a is provided with one or more buttonholes 111a at the bottom end thereof. The detachable means 110 like the buttons 111b and the buttonholes 111a are of course placed to the opposite position to each other in accordance with the type of clothes coupled to the cover body 50a.

Additionally, the cover body 50a is provided with a pocket 56 in which items are put at one side surface thereof, which helps the wearer who works at night or outside easily carry his items necessary to work.

Especially, the items the wearer uses every day like a cellular phone, coins, keys, and the like are put in the pocket 56, and the pocket 56 is provided with locking means 56a at the inner side of the top portion thereof, for preventing the items therein from dropping to the outside. The locking means 56a is formed of a pair of Velcro tape, a pair of snaps, a zipper, or the like.

FIG. 15 shows still another embodiment of the present invention where the cover body 50a is coupled to the top side of panties 102. The detachable means 110 is formed of a pair of snaps 112a and 112b that are placed at the top end of the panties 102 and at the bottom end of the cover body 50a. At this time, the fastening band 60 is coupled at one side of the cover body 50a by means of the button 61a and the buttonhole 61a, and otherwise, the fastening bands 60 are coupled at both sides of the cover body 50a in such a manner as to be tied to each other.

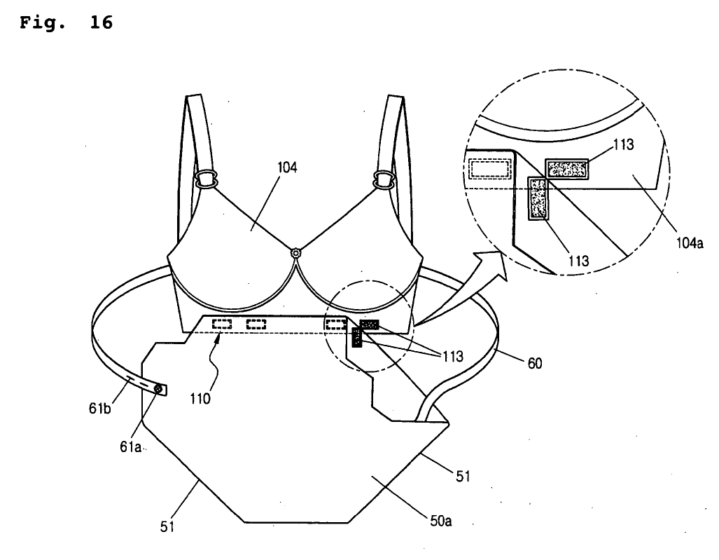

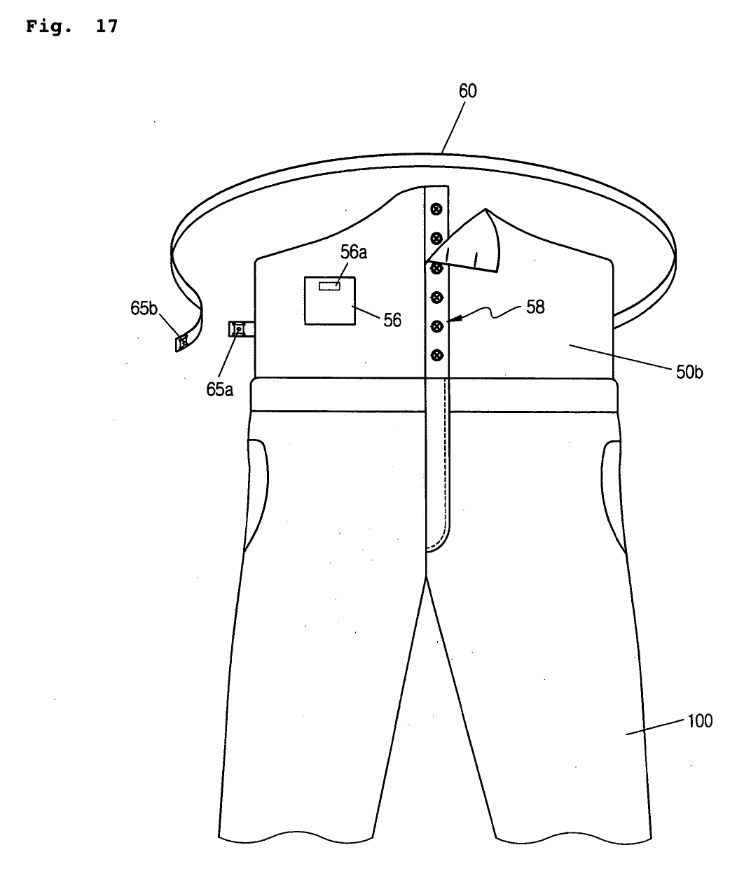

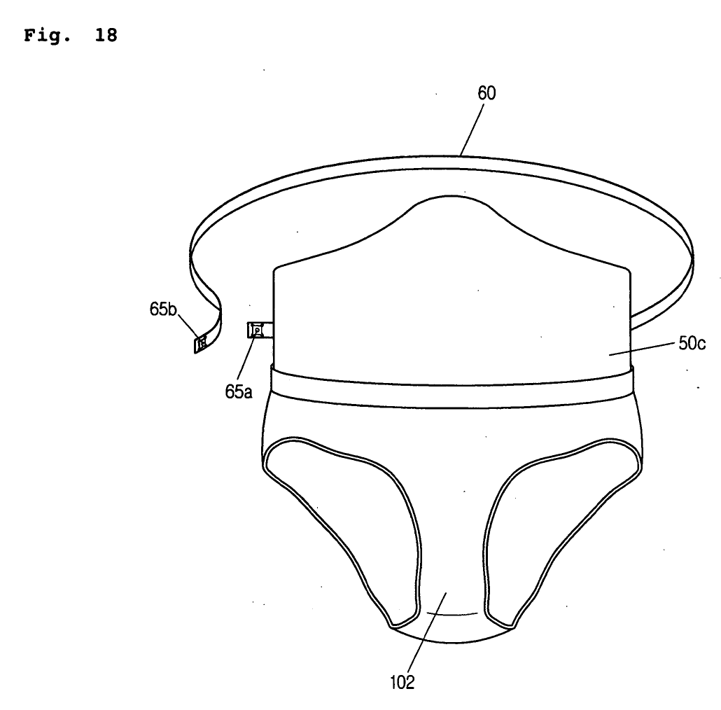

FIG. 16 shows another embodiment of the present invention where the cover body 50a is coupled to a brassiere 104 as a kind of women's underwear. As shown, a pair of Velcro tape 113 are provided at an extended portion 104a of the brassiere 104 and at the top end of the cover body 50a. Thereby, when the underwear like panties and brassiere that is coupled to the cover body 50a is worn, the wearer's abdominal region can be covered and protected. In this case, also, there is no inconvenience occurring when the abdomen cover should be worn apart from the underwear before the wearer goes to bed.

The detachable means 110 of the various kinds of clothes and the abdomen cover is embodied with another means like hooks, a toggle button and a loop, a zipper, or the like, and the kinds of clothes include skirts, shirts, pajamas, vests, all kinds of special clothes such as operating gowns, patient gowns, working clothes, mountain-climbing clothes, traditional clothes, suits, school uniforms, and so on.

According to still another embodiment of the present invention, there is provided an abdomen cover including: a cover body 50b coupled integrally to clothes at one side thereof; and a fastening band 60 formed at the end portion of one side or each of both sides of the cover body 50b.

In other words, unlike the embodiments where the cover body 50b is detachably coupled to all kinds of clothes, the abdomen cover of this invention is formed integrally with the clothes to be manufactured, such that when the clothes are worn, the wearer's abdominal region can be covered and protected.

The cover body 50b is designed to cover the wearer's abdominal region, and thus, it is formed integrally only with the front of the pants.

The cover body 50b is cut along a longitudinal direction at a central portion thereof such that it is openable to the left and right sides, and the cover body 50b is provided with locking means 58 at the central portion thereof.

Like the coupling means of the fastening band 60, the detachable means 110 of the cover body 50a and the clothes, and the locking means 56a of the pocket 56, the locking means include buttons, snaps, hooks, Velcro tape, a zipper, and the like. Therefore, in the state where the abdomen cover is worn together with the clothes the central portion of the cover body 50b is freely opened by means of the locking means 58.

In addition, the cover body 52b that is formed integrally with the clothes is provided with a pocket 56 in which items are put at one side thereof.

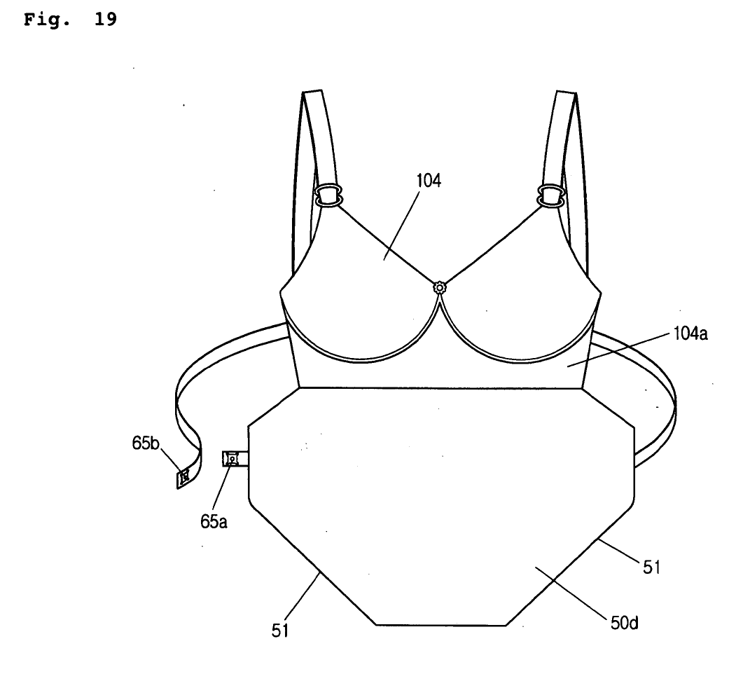

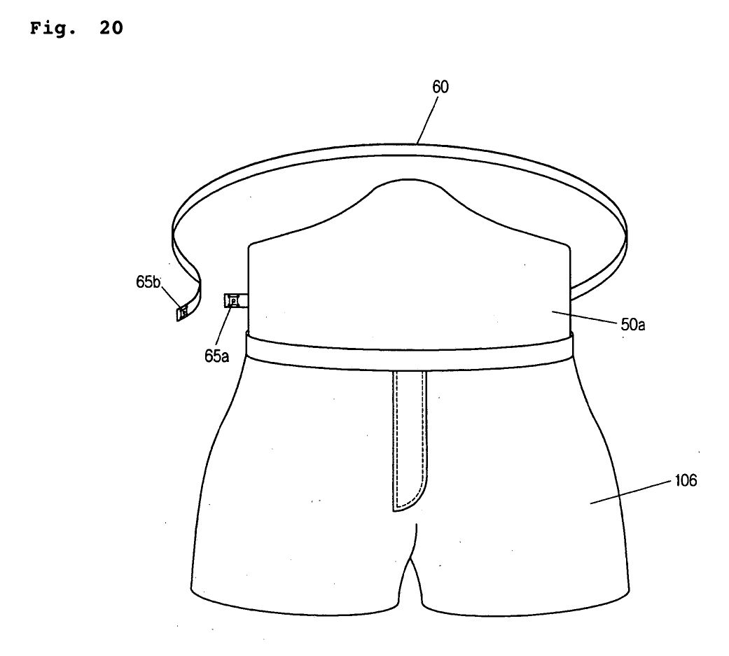

FIG. 18 shows another embodiment of the present invention where a cover body 50c is formed integrally with panties 102, and FIG. 19 shows yet another embodiment of the present invention where a cover body 50d is formed integrally with a downward extended portion 104a of a brassiere 104.

FIG. 20 shows still another embodiment of the present invention where the cover body 50a is formed integrally with the top portion of short pants (or trunk panties) 106.

As set forth in the foregoing, the abdomen cover according to the present invention is adapted to cover a wearer's abdominal region for keeping his abdominal region at a warm state, thereby preventing the generation of trouble in his abdomen and to fit the fastening band that is formed at one side end portion of the cover body around a wearer's waist in such a manner as to be detachably coupled to the other side end portion thereof, thereby making the wearer put on the abdomen cover in an easy manner.

In addition, the abdomen cover of this invention prevents the movement of the cover body when in use and with the formation of the auxiliary support band, also prevents the side thereof from being separated from the wearer's abdominal region.

Besides, the cover body has the inclined portion fit for the human body at the both sides thereof, respectively, thereby improving a feeling of wearing, and the abdomen cover of this invention is formed integrally with all kinds of clothes or detachably coupled to them.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1-29. (canceled)

30. An abdomen cover adapted to cover a wearer's abdomen region to protect the wearer's abdomen region, comprising:
a cover body having an inclined portion slantingly formed at both sides thereof to be conform to a wearer's body shape;
an extended portion having a gently curved shape formed at the upper or lower portion of the cover body in such a manner as to be protruded upwardly or downwardly from the upper or lower portion of the cover body;
a heart-shaped or “ [symbol] ” or angle concave portion formed at the extended portion of the upper portion of the cover body; and
a fastening band formed at the end portion of one side or each of both sides of the cover body, for tying the cover body around the wearer's abdominal region.

31. The abdomen cover according to claim 30, wherein the extended portion of the lower portion of the cover body has a heart-shaped or “ [symbol] ” or angle concave portion formed thereon to be opposed to the heart-shaped or “ [symbol] ” or angle concave portion formed at the extended portion of the upper portion of the cover body.

32. The abdomen cover according to claim 30, wherein the extended portion formed at the lower portion of the cover body is extended downwardly to cover the wearer's groin and private region.

33. The abdomen cover according to claim 31, wherein the extended portion formed at the lower portion of the cover body is extended downwardly to cover the wearer's groin and private region.

34. An abdomen cover according to claim 30, wherein the fastening band includes a single band integrally coupled to the cover body.

35. An abdomen cover according to claim 30, wherein the fastening band or the cover body has any one of a detachable Velcro tape, a snap and a hook which are formed at the end portion thereof in such a manner as to have a male and female pair.

36. An abdomen cover according to claim 30, wherein the fastening band and the cover body are provided with one or more buttons and buttonholes that are fastened to each other at the end portion thereof.

37. An abdomen cover according to claim 36, wherein the button is a toggle button, and the fastening band or the cover body is provided with a coupling loop into which the toggle button is fastened, at the end portion thereof.

38. An abdomen cover according to claim 30, wherein the fastening band is formed of any one of fabric, a rubber material, and a mixture of the fabric with the rubber material.

39. An abdomen cover according to claim 30, wherein the fastening band is covered with the fabric around an outer surface thereof and is embedded with the rubber material in the inside thereof.

40. An abdomen cover according to claim 39, wherein the fastening band has a folded portion provided on the outer surface thereof, for applying an elastic force thereto.

41. An abdomen cover according to claim 30, wherein the cover body is provided with a pair of auxiliary support bands at one side or each of the both end portions thereof in such a manner as to be met and coupled to the fastening band.

42. An abdomen cover according to claim 41, wherein the auxiliary support band is coupled to the edge portion of the cover body at one end thereof in such a manner as to be coupled to the fastening band.

43. An abdomen cover according to claim 30, wherein the cover body is provided with an edge portion member along a circumferential direction thereof.

44. An abdomen cover according to claim 30, wherein the cover body is provided with a heating member in the inside thereof.

45. An abdomen cover according to claim 44, wherein the heating member includes any one of cotton, fabric, and a plant.

46. An abdomen cover according to claim 30, wherein the cover body is provided with a pocket at one face thereof.

47. An abdomen cover according to claim 46, wherein the pocket has locking means formed at the inside of the top portion thereof.

48. An abdomen cover according to claim 47, wherein the locking means has any one of a detachable Velcro tape, a snap, a button and a zipper which are formed in such a manner as to have a male and female pair.

49. An abdomen cover according to claim 38, wherein the fastening band is covered with the fabric around an outer surface thereof and is embedded with the rubber material in the inside thereof.
